(19) United States
(12) Patent Application Publication
(10) Pub. No.: US 2022/0079600 A1
(43) Pub. Date: Mar. 17, 2022

(54) MEDICAL GUIDEWIRE ASSEMBLY

(71) Applicant: Baylis Medical Company Inc., Montreal (CA)

(72) Inventors: Eduardo Moriyama, Richmond (CA); Carline Quach, Concord (CA)

(21) Appl. No.: 17/411,149

(22) Filed: Aug. 25, 2021

Related U.S. Application Data
(60) Provisional application No. 63/078,563, filed on Sep. 15, 2020.

(51) Int. Cl. A61B 17/12; A61B 1/04; A61B 1/06; A61M 31/00

(52) U.S. Cl. CPC A61B 17/12013; A61B 1/043; A61B 2017/00243; A61M 31/005; A61B 1/0653

(57) ABSTRACT

A method is used for closing, and confirming closure of, the left atrial appendage of the heart of a patient. The method includes using an assembly for closing the left atrial appendage. The method also includes using a system for confirming closure of the left atrial appendage after the left atrial appendage has been closed by the assembly.

### TECHNICAL FIELD

This document relates to the technical field of (and is not limited to) a medical guidewire assembly configured to be attracted to another medical guidewire assembly (and method therefor).

### BACKGROUND

Known medical devices are configured to facilitate a medical procedure, and help healthcare providers diagnose and/or treat medical conditions of sick patients.

### SUMMARY

It will be appreciated that there exists a need to mitigate (at least in part) at least one problem associated with existing medical guidewires (also called the existing technology). After much study of, and experimentation with, the existing medical guidewires, an understanding (at least in part) of the problem and its solution have been identified (at least in part) and are articulated (at least in part) as follows:

In patients with non-valvular atrial fibrillation, stroke-causing clots may form in the left atrial appendage (LAA). Closing the left atrial appendage through a procedure called left atrial appendage ligation (LAAL) may reduce the risk of stroke in these patients. During LAAL, devices, such as the LARIAT system, may utilize an epicardial approach to externally ligate the left atrial appendage and permanently seal the left atrial appendage off from the rest of the heart. This procedure prevents blood from circulating through and pooling in the left atrial appendage and causing clots, potentially decreasing the risk of stroke. Once ligated (closed), the left atrial appendage atrophies due to lack of blood circulation. However, if the blood supply is not completely stopped, the patient may require a new surgery to reduce their risk of stroke. To ensure complete lack of blood circulation, fluorescence angiography (using infrared imaging via an epicardial approach) may be used.

To mitigate, at least in part, at least one problem associated with the existing technology, there is provided (in accordance with a major aspect) a method. The method is for closing, and confirming the closing of, the left atrial appendage of the heart of a patient. The method includes and is not limited to (comprises) using an assembly for closing the left atrial appendage. The method also includes using a system for confirming the closure of the left atrial appendage after the left atrial appendage has been closed by the assembly.

To mitigate, at least in part, at least one problem associated with the existing technology, there is provided (in accordance with another major aspect) a method. The method is for closing, and confirming the closing of, the left atrial appendage of the heart of a patient. The method includes and is not limited to (comprises) using a lariat assembly for closing (ligating) the left atrial appendage. The method also includes using an epicardial fluorescence angiogram system for confirming the closure of the left atrial appendage after the left atrial appendage has been closed by the epicardial device.

Other aspects are identified in the claims. Other aspects and features of the non-limiting embodiments may now become apparent to those skilled in the art upon review of the following detailed description of the non-limiting embodiments with the accompanying drawings. This Summary is provided to introduce concepts in simplified form that are further described below in the Detailed Description. This Summary is not intended to identify potentially key features or possible essential features of the disclosed subject matter, and is not intended to describe each disclosed embodiment or every implementation of the disclosed subject matter. Many other novel advantages, features, and relationships will become apparent as this description proceeds. The figures and the description that follow more particularly exemplify illustrative embodiments.

### BRIEF DESCRIPTION OF THE DRAWINGS

The non-limiting embodiments may be more fully appreciated by reference to the following detailed description of the non-limiting embodiments when taken in conjunction with the accompanying drawings, in which:

FIG. 1 depicts a side cross-sectional view of an embodiment of a patient, a first medical guidewire assembly and a second medical guidewire assembly; and

FIG. 2 depicts a side cross-sectional view of an embodiment of the patient, the first medical guidewire assembly and the second medical guidewire assembly of FIG. 1; and

FIG. 3, FIG. 4 and FIG. 5 depict close-up side cross-sectional views of embodiments of the patient, the first medical guidewire assembly and the second medical guidewire assembly of FIG. 1; and

FIG. 6 depicts a schematic view of an embodiment of FIG. 1; and

FIG. 7 depicts a side cross-sectional view of an embodiment of the patient of FIG. 1.

The drawings are not necessarily to scale and may be illustrated by phantom lines, diagrammatic representations and fragmentary views. In certain instances, details unnecessary for an understanding of the embodiments (and/or details that render other details difficult to perceive) may have been omitted. Corresponding reference characters indicate corresponding components throughout the several figures of the drawings. Elements in the several figures are illustrated for simplicity and clarity and have not been drawn to scale. The dimensions of some of the elements in the figures may be emphasized relative to other elements for facilitating an understanding of the various disclosed embodiments. In addition, common, and well-understood, elements that are useful in commercially feasible embodiments are often not depicted to provide a less obstructed view of the embodiments of the present disclosure.

### LISTING OF REFERENCE NUMERALS USED IN THE DRAWINGS

first medical guidewire assembly 101
first distal magnetic flux emitter 106
second medical guidewire assembly 202
second distal magnetic flux emitter 206
lariat assembly 300
expandable device 400
catheter 500
sensor device 502
first sensor view 511
second sensor view 512
third sensor view 513
biological feature 904
first side 901
second side 902
patient 908
heart 910
suture 912

### DETAILED DESCRIPTION OF THE NON-LIMITING EMBODIMENT(S)

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the disclosure is defined by the claims. For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Technical Field, Background, Summary or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the disclosure is limited to the subject matter provided by the claims, and that the disclosure is not limited to the particular aspects depicted and described. It will be appreciated that the scope of the meaning of a device configured to be coupled to an item (that is, to be connected to, to interact with the item, etc.) is to be interpreted as the device being configured to be coupled to the item, either directly or indirectly. Therefore, “configured to” may include the meaning “either directly or indirectly” unless specifically stated otherwise.

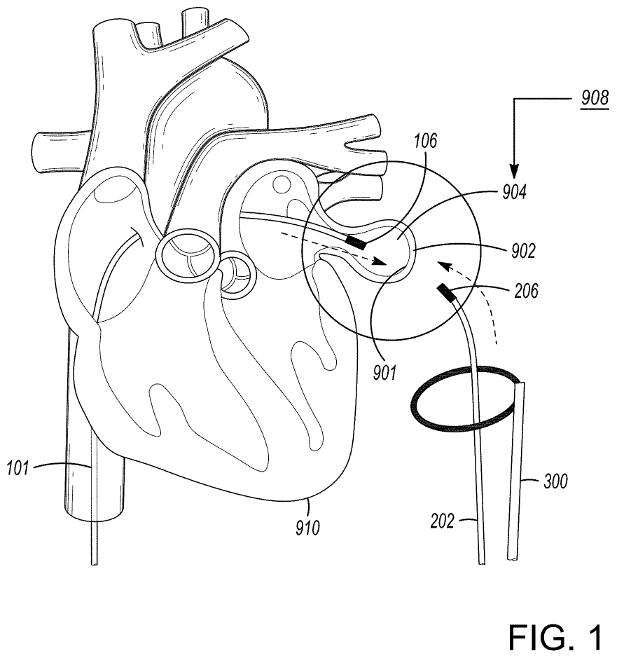

FIG. 1 depicts a side cross-sectional view of an embodiment of a patient 908, a first medical guidewire assembly 101 and a second medical guidewire assembly 202.

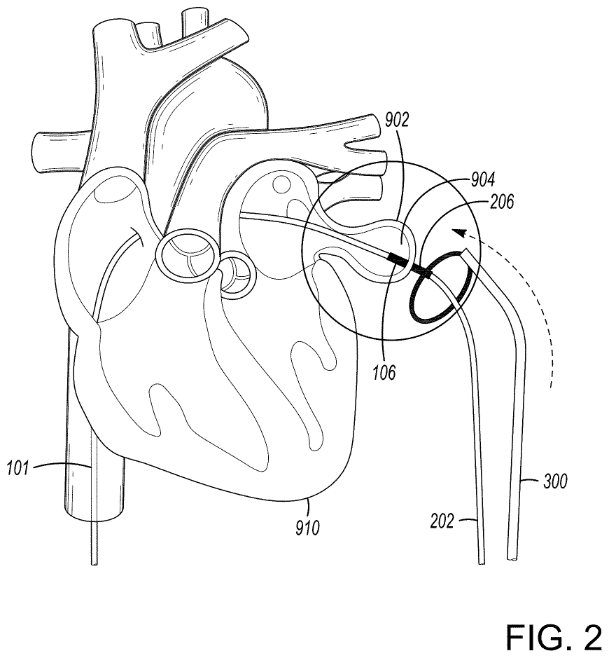

FIG. 2 depicts a side cross-sectional view of an embodiment of the patient 908, the first medical guidewire assembly 101 and the second medical guidewire assembly 202 of FIG. 1.

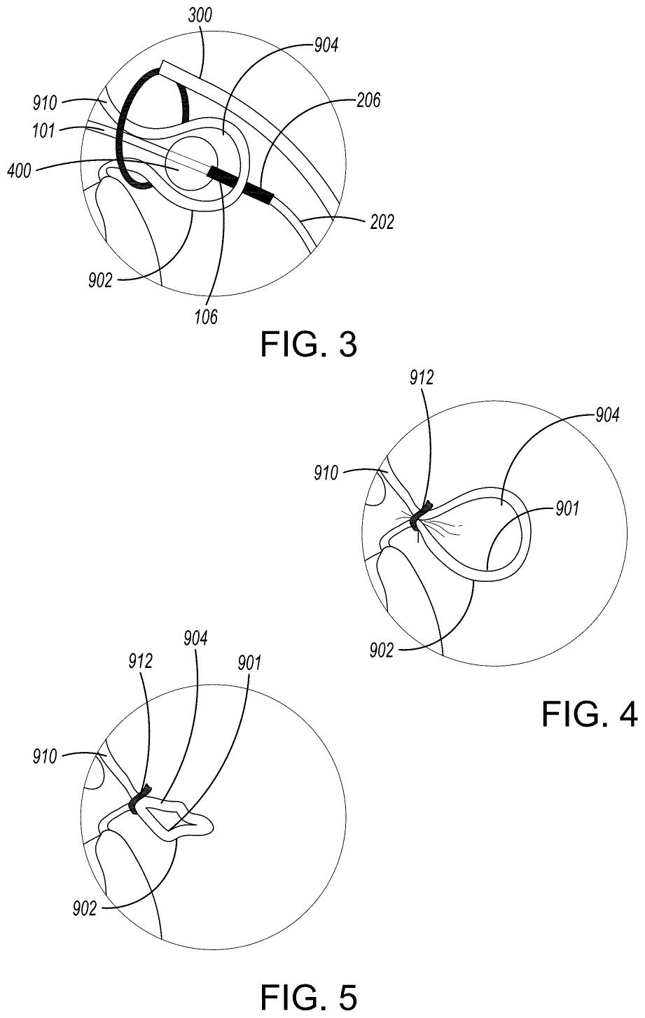

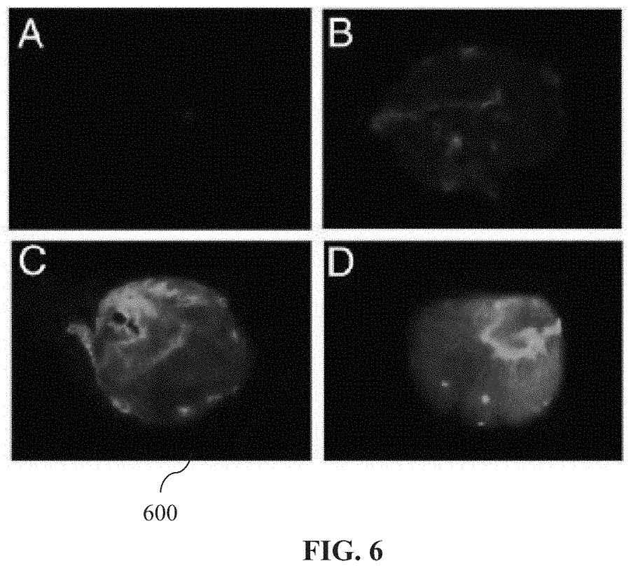

Referring to the embodiments (implementations) as depicted in FIG. 1 and FIG. 2, the patient 908 has a biological feature 904 with a first side 901 and a second side 902. The biological feature 904 includes, for instance, a left atrial appendage of (extending from) the heart 910 of the patient 908, in which the left atrial appendage is depicted as being inflamed. A procedure is utilized to treat the left atrial appendage (as depicted in FIG. 2 or FIG. 3) in such a way that the left atrial appendage may become atrophied (as depicted in FIG. 6).

Referring to the embodiment (implementation) as depicted in FIG. 1, the first medical guidewire assembly 101 is configured to be positioned proximate to the first side 901 of the biological feature 904. The second medical guidewire assembly 202 is configured to be positioned proximate to the second side 902 of the biological feature 904. The second medical guidewire assembly 202 is also configured to be attracted to the first medical guidewire assembly 101.

Referring to the embodiment (implementation) as depicted in FIG. 1, the first medical guidewire assembly 101, preferably, has a first distal magnetic flux emitter 106 configured to be positioned proximate to the first side 901 of the biological feature 904. The second medical guidewire assembly 202, preferably, has a second distal magnetic flux emitter 206 configured to be positioned proximate to the second side 902 of the biological feature 904. The second distal magnetic flux emitter 206 is also configured to be magnetically attracted to the first distal magnetic flux emitter 106. The second distal magnetic flux emitter 206 is, preferably, configured to be magnetically attracted to the first distal magnetic flux emitter 106; this is done, preferably, in such a way that the first distal magnetic flux emitter 106 and the second distal magnetic flux emitter 206, in use, securely hold, at least in part, the biological feature 904 (after the first distal magnetic flux emitter 106 and the second distal magnetic flux emitter 206 have become, respectively, positioned proximate to the first side 901 of the biological feature 904 and the second side 902 of the biological feature 904).

Referring to the embodiment (implementation) as depicted in FIG. 1, the first medical guidewire assembly 101 has a first distal magnetic flux emitter 106 configured to emit magnetic flux, and also configured to be inserted, at least in part, into the patient 908, and positioned proximate to the first side 901 of the biological feature 904. The second medical guidewire assembly 202 has a second distal magnetic flux emitter 206 configured to emit magnetic flux, and also configured to be inserted, at least in part, into the patient 908, and positioned proximate to the second side 902 of the biological feature 904. The second distal magnetic flux emitter 206 is also configured to be magnetically attracted to the first distal magnetic flux emitter 106; this is done, preferably, in such a way that the first distal magnetic flux emitter 106 and the second distal magnetic flux emitter 206, in use, securely hold, at least in part, the portion of the biological feature 904 between the first distal magnetic flux emitter 106 and the second distal magnetic flux emitter 206 (preferably, after the first distal magnetic flux emitter 106 and the second distal magnetic flux emitter 206 have become respectively positioned proximate to the first side 901 of the biological feature 904 and the second side 902 of the biological feature 904). For instance, the first distal magnetic flux emitter 106 includes a first magnetic element. For instance, the second distal magnetic flux emitter 206 includes a second magnetic element. For instance, the first distal magnetic flux emitter 106 includes a first magnetic element and the second distal magnetic flux emitter 206 includes a second magnetic element. For instance, the first distal magnetic flux emitter 106 includes a first solenoid element. For instance, the second distal magnetic flux emitter 206 includes a second solenoid element. For instance, the first distal magnetic flux emitter 106 includes a first solenoid element and the second distal magnetic flux emitter 206 includes a second solenoid element.

Referring to the embodiment (implementation) as depicted in FIG. 1, a distal portion of the first medical guidewire assembly 101 is inserted into the patient 908. The first distal magnetic flux emitter 106 is mounted to the distal portion of the first medical guidewire assembly 101. Specifically, the distal portion of the first medical guidewire assembly 101 is inserted into the interior of the heart 910. The distal portion of the first medical guidewire assembly 101 is maneuvered through the heart 908 and into the biological feature 904 (left atrial appendage), toward and into the interior of the biological feature 904 (that is, moved toward the first side 901 of the biological feature 904). The first side 901 (an interior surface) is located within the interior of the biological feature 904.

Referring to the embodiment (implementation) as depicted in FIG. 1, a distal portion of the second medical guidewire assembly 202 is inserted into the patient 908, but is positioned (and is maneuvered) exteriorly of the heart 910. The second distal magnetic flux emitter 206 is mounted to the distal portion of the second medical guidewire assembly 202. The distal portion of the second medical guidewire assembly 202 is maneuvered toward the exterior of the biological feature 904 (such as the left atrial appendage). The second medical guidewire assembly 202 is moved toward the second side 902 of the biological feature 904. The second side 902 (an exterior surface) is located on the exterior of the biological feature 904.

Referring to the embodiment (implementation) as depicted in FIG. 1, a lariat assembly 300 is configured to be moved along and guided by the second medical guidewire assembly 202 toward the biological feature 904 (such as, the left atrial appendage). The lariat assembly 300 is configured to cause the biological feature 904 (such as, the left atrial appendage) to become atrophied after the lariat assembly 300 is installed to the biological feature 904 and is activated accordingly. The lariat assembly 300 is configured to perform LAAL. A lariat is a rope in the form of a lasso. The lariat assembly 300 includes biocompatible material properties suitable for specific performance (such as, dielectric strength, thermal, electrical insulation, corrosion, water resistance, heat resistance, etc.), for compliance with industrial and regulatory safety standards (or compatible for medical usage), etc. Reference is made to the following publication for consideration in the selection of a suitable material: Plastics in Medical Devices: Properties, Requirements, and Applications; 2nd Edition; author: Vinny R. Sastri; hardcover ISBN: 9781455732012; published: 21 Nov. 2013; publisher: Amsterdam [Pays-Bas]: Elsevier/William Andrew, [2014].

Referring to the embodiment (implementation) as depicted in FIG. 2, the first distal magnetic flux emitter 106 (of the first medical guidewire assembly 101) and the second distal magnetic flux emitter 206 (of the second medical guidewire assembly 202) are moved toward each other, and make contact with each other. The first distal magnetic flux emitter 106 and the second distal magnetic flux emitter 206 are magnetically attracted to each other; this is done in such a way that a portion of the biological feature 904 may become securely held into a relatively stationary position. After the biological feature 904 is securely held into a relatively stationary position, the lariat assembly 300 is moved toward the biological feature 904, and the lariat assembly 300 may be positioned for secured connection to a section of the biological feature 904.

Referring to the embodiments (implementations) as depicted in FIG. 2, epicardial access may be achieved by using a mechanical needle to puncture the pericardium (while avoiding the myocardium). The lariat assembly 300 may utilize, if desired, a number 21G needle assembly (known and not depicted) followed by the placement of a number 13F guide cannula assembly (known and not depicted). Epicardial access may include, for instance, the application of an input force to a mechanical needle, etc.

Referring to the embodiments (implementations) as depicted in FIG. 2, the lariat assembly 300 may include any type of device configured to snare a portion of the biological feature 904, and to deliver and install a suture, etc. For instance, the lariat assembly 300 may be used with magnetically tipped guidewires that are attached endocardially and epicardially to create a rail (once they are attached or magnetically attached in an end-to-end relationship). The lariat assembly 300 is configured to use the rail to advance to the closure location; this arrangement may be identified by deployment of the expandable device 400 (such as an occlusion balloon, as depicted in FIG. 3, if so desired). The lariat assembly 300 is configured to (A) close the biological feature 904, and/or (B) to release (install) a suture 912 (as depicted in FIG. 4), and tighten the suture 912. The expandable device 400 includes biocompatible material properties suitable for specific performance (such as, dielectric strength, thermal, electrical insulation, corrosion, water resistance, heat resistance, etc.), for compliance with industrial and regulatory safety standards (or compatible for medical usage), etc. Reference is made to the following publication for consideration in the selection of a suitable material: Plastics in Medical Devices: Properties, Requirements, and Applications; 2nd Edition; author: Vinny R. Sastri; hardcover ISBN: 9781455732012; published: 21 Nov. 2013; publisher: Amsterdam [Pays-Bas]: Elsevier/William Andrew, [2014].

FIG. 3, FIG. 4 and FIG. 5 depict close-up side cross-sectional views of embodiments of the patient 908, the first medical guidewire assembly 101 and the second medical guidewire assembly 202 of FIG. 1.

Referring to the embodiment (implementation) as depicted in FIG. 3, the first distal magnetic flux emitter 106 (of the first medical guidewire assembly 101) and the second distal magnetic flux emitter 206 (of the second medical guidewire assembly 202) continue to securely hold the biological feature 904 in a relatively stationary position. An expandable device 400 (such as, a balloon, etc., and any equivalent thereof) is installed to the distal section of the first medical guidewire assembly 101. Before the lariat assembly 300 is further moved and positioned for secured connection to a section of the biological feature 904, the expandable device 400 is activated (inflated or expanded); this is done in such a way that the interior of the biological feature 904 may be supported by the expandable device 400. The expandable device 400 is deployed to ensure that the lariat assembly 300 may be positioned for optimal deployment (that is, installment of a lariat to a desired portion of the biological feature 904).

Referring to the embodiment (implementation) as depicted in FIG. 3, the first distal magnetic flux emitter 106 (of the first medical guidewire assembly 101) and the second distal magnetic flux emitter 206 (of the second medical guidewire assembly 202) continue to securely hold the biological feature 904 in a relatively stationary position. After the expandable device 400 is activated (inflated or expanded), the interior of the biological feature 904 is supported by the expandable device 400, and the lariat assembly 300 may be further positioned for secured connection to the desired section of the biological feature 904. After the lariat assembly 300 is moved and positioned, as depicted, the lariat assembly 300 may be activated (that is, for the installment of a lariat to a desired portion of the biological feature 904).

Referring to the embodiment (implementation) as depicted in FIG. 4, the lariat assembly 300 has been activated to apply an atrophication element 912 (such as, a suture, etc.) that causes the biological feature 904 (depicted as the left atrial appendage) to become pinched, thereby initiating atrophication of the biological feature 904. After the atrophication element 912 is installed to the biological feature 904, the lariat assembly 300 may be retracted or withdrawn. After the atrophication element 912 has been installed, the expandable device 400 may be deactivated (deflated). The lariat assembly 300 may be retracted away from the biological feature 904. The first medical guidewire assembly 101 and the second medical guidewire assembly 202 may be retracted away from the biological feature 904. Separation movement between the first medical guidewire assembly 101 and the second medical guidewire assembly 202 overcomes the magnetic attraction between the first distal magnetic flux emitter 106 (of the first medical guidewire assembly 101) and the second distal magnetic flux emitter 206 (of the second medical guidewire assembly 202).

Referring to the embodiment (implementation) as depicted in FIG. 5, the atrophication element 912 has caused the biological feature 904 (depicted as the left atrial appendage) to become completely atrophied.

FIG. 6 depicts a schematic view of an embodiment of the patient 908 of FIG. 1.

Referring to the embodiments (implementations) as depicted in FIG. 6, an epicardial fluorescence angiogram system 600 is configured to detect the presence of an indocyanine green dye injected proximate to the biological feature 904, for confirmation that the biological feature 904 has been closed (as depicted in FIG. 5). It will be appreciated that any medical imaging technique may be utilized to confirm closure of the biological feature 904. For example, transthoracic echocardiography, transesophageal echocardiography, intracardiac echocardiography, cardiac computed tomography and cardiac magnetic resonance imaging have been described in literature as other possible imaging tools.

Referring to the embodiments (implementations) as depicted in FIG. 6, there is depicted an example of fluorescence angiography of the closure of the biological feature 904 in which it is determined that, based on the medical image of fluorescence angiography, there is blood flow in the biological feature 904. For this case, closure of the biological feature 904 was not performed successfully.

Referring to the embodiments (implementations) as depicted in FIG. 1 to FIG. 6, the procedural workflow may include the following steps (of a method), as follows: step (A) includes gaining epicardial access (as depicted in FIG. 1); step (B) includes performing LAAL or the left atrial appendage ligation operation (that is, closing the left atrial appendage) (as depicted in FIG. 3); step (C) includes introducing or injecting (preferably proximate to the left atrial appendage, the vasculature, etc.) a quantity of dye configured to be detectable by a fluorescence endoscopic system (as depicted in FIG. 6); step (D) includes turning on (activating) the excitation light of the fluorescence endoscopic system (as depicted in FIG. 6); and step (E) includes watching for the dye to display areas (of the vasculature) having normal blood flow so that the closure of the left atrial appendage may be confirmed by a lack of fluorescence emanating from the left atrial appendage (as depicted in FIG. 6).

Referring to the embodiments (implementations) as depicted in FIG. 1 to FIG. 6, there is provided a method for closing, and confirming the closing of, the left atrial appendage of the heart of a patient. The method includes using a lariat assembly 300 for closing (ligating) the left atrial appendage. The method also includes using an epicardial fluorescence angiogram system 600 for confirming the closure of the left atrial appendage after the left atrial appendage has been closed by the epicardial device. The method may be adapted such that the step of using an epicardial fluorescence angiogram system includes introducing a fluorescence endoscopic dye, via an epicardial approach, at a position located proximate to left atrial appendage. The method may be adapted such that the step of using an epicardial fluorescence angiogram system includes activating the fluorescence excitation light after the fluorescence endoscopic dye (such as indocyanine green, fluorescein, methylene blue or other fluorescentdye) has been introduced. The method may be adapted such that the step of using an epicardial fluorescence angiogram system includes detecting a presence of fluorescence associated with the fluorescence endoscopic dye positioned in the areas with normal blood flow in the heart. The method may be adapted such that the step of using an epicardial fluorescence angiogram system includes detecting a lack of fluorescence associated with the lack of the fluorescence endoscopic dye positioned in the left atrial appendage, which provides confirmation of the closure of the left atrial appendage.

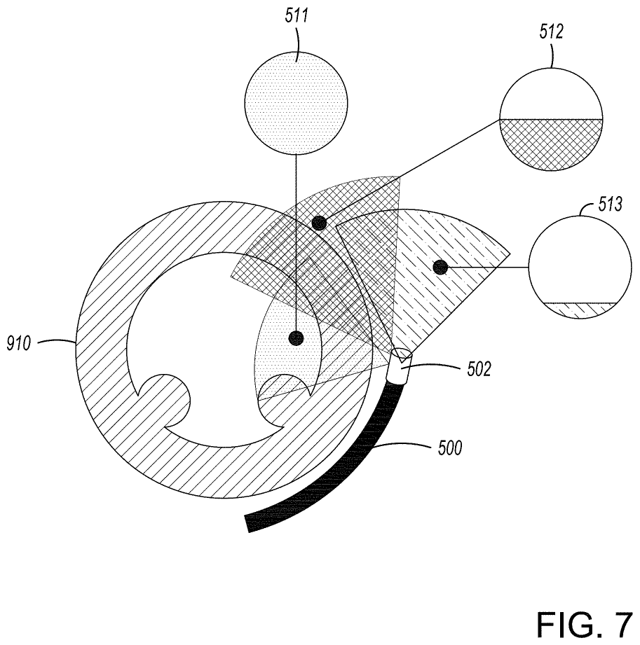

FIG. 7 depicts a side cross-sectional view of an embodiment of the patient 908 of FIG. 1.

Referring to the embodiment (implementation) as depicted in FIG. 7, during the procedure, a distal portion of a catheter 500 may be deployed and positioned proximate to the heart 910 of the patient, in close proximity to the biological feature 904 (as depicted in FIG. 2). A sensor device 502 (such as, a camera device, etc.) is mounted to the distal portion of the catheter 500. The sensor device 502 is configured to provide confirmation information (visual information) to the physician as to whether the first distal magnetic flux emitter 106 (of the first medical guidewire assembly 101) and the second distal magnetic flux emitter 206 (of the second medical guidewire assembly 202) are positioned accordingly for securing the biological feature 904, and are also magnetically attracted to each other; this is done, preferably, before the lariat assembly 300 is positioned proximate to the biological feature 904 and activated, and before the expandable device 400 is activated. The sensor device 502 is configured, preferably, to provide confirmation information in the form of a first sensor view 511, a second sensor view 512 and a third sensor view 513, depending on the spatial orientation of the sensor device 502 relative to the biological feature 904. The catheter 500 and the sensor device 502 include biocompatible material properties suitable for specific performance (such as, dielectric strength, thermal, electrical insulation, corrosion, water resistance, heat resistance, etc.), for compliance with industrial and regulatory safety standards (or compatible for medical usage), etc. Reference is made to the following publication for consideration in the selection of a suitable material: Plastics in Medical Devices: Properties, Requirements, and Applications; 2nd Edition; author: Vinny R. Sastri; hardcover ISBN: 9781455732012; published: 21 Nov. 2013; publisher: Amsterdam [Pays-Bas]: Elsevier/William Andrew, [2014].

Referring to the embodiments (implementations) as depicted in FIG. 7, a steerable pericardial endoscope may be used. The direction of the objective lens may be, preferably, in the correct visual field. A floppy sheath may be used to prevent compression of the heart surface and deterioration of hemodynamic parameters.

Referring to the embodiments (implementations) as depicted in FIG. 7, the sensor device 502 may be included in an endoscope system having a light source, camera (micro camera) and one or more optical filters. The light source may illuminate at the excitation wavelength of ICG (indocyanine green, about 800 nm). The light source may include Light-Emitting Diodes (LEDs), a halogen lamp, a diode laser, etc. The micro camera may include a near infrared (NIR) camera. The optical filter may prevent the mixing of the fluorescing (weak) and excitation (strong) rays that sum at the camera sensor. More specifically, a high-pass filter may be used for filtering wavelengths going to the source (blood & ICG) and a low-pass filter may be used for filtering wavelengths going to the camera. The optical filter may be configured to block the light wavelengths for fluorescence. ICG (indocyanine green) imaging techniques may be used for infrared fluorescence dye, or a dye for infracyanine green (IfCG) may be used, etc.

Referring to the embodiments (implementations) as depicted in FIG. 2 to FIG. 7, the procedure, as described, may be used to confirm the closure of other cardiac structures, such as veins and arteries, etc.

The following is offered as further description of the embodiments, in which any one or more of any technical feature (described in the detailed description, the summary and the claims) may be combinable with any other one or more of any technical feature (described in the detailed description, the summary and the claims). It is understood that each claim in the claims section is an open ended claim unless stated otherwise. Unless otherwise specified, relational terms used in these specifications should be construed to include certain tolerances that the person skilled in the art would recognize as providing equivalent functionality. By way of example, the term perpendicular is not necessarily limited to 90.0 degrees, and may include a variation thereof that the person skilled in the art would recognize as providing equivalent functionality for the purposes described for the relevant member or element. Terms such as “about” and “substantially”, in the context of configuration, relate generally to disposition, location, or configuration that are either exact or sufficiently close to the location, disposition, or configuration of the relevant element to preserve operability of the element within the disclosure which does not materially modify the disclosure. Similarly, unless specifically made clear from its context, numerical values should be construed to include certain tolerances that the person skilled in the art would recognize as having negligible importance as they do not materially change the operability of the disclosure. It will be appreciated that the description and/or drawings identify and describe embodiments of the apparatus (either explicitly or inherently). The apparatus may include any suitable combination and/or permutation of the technical features as identified in the detailed description, as may be required and/or desired to suit a particular technical purpose and/or technical function. It will be appreciated that, where possible and suitable, any one or more of the technical features of the apparatus may be combined with any other one or more of the technical features of the apparatus (in any combination and/or permutation). It will be appreciated that persons skilled in the art would know that the technical features of each embodiment may be deployed (where possible) in other embodiments even if not expressly stated as such above. It will be appreciated that persons skilled in the art would know that other options may be possible for the configuration of the components of the apparatus to adjust to manufacturing requirements and still remain within the scope as described in at least one or more of the claims. This written description provides embodiments, including the best mode, and also enables the person skilled in the art to make and use the embodiments. The patentable scope may be defined by the claims. The written description and/or drawings may help to understand the scope of the claims. It is believed that all the crucial aspects of the disclosed subject matter have been provided in this document. It is understood, for this document, that the word “includes” is equivalent to the word “comprising” in that both words are used to signify an open-ended listing of assemblies, components, parts, etc. The term “comprising”, which is synonymous with the terms “including,” “containing,” or “characterized by,” is inclusive or open-ended and does not exclude additional, unrecited elements or method steps. Comprising (comprised of) is an “open” phrase and allows coverage of technologies that employ additional, unrecited elements. When used in a claim, the word “comprising” is the transitory verb (transitional term) that separates the preamble of the claim from the technical features of the disclosure. The foregoing has outlined the non-limiting embodiments (examples). The description is made for particular non-limiting embodiments (examples). It is understood that the non-limiting embodiments are merely illustrative as examples.

What is claimed is:

1. A method for closing, and confirming closure of, the left atrial appendage of the heart of a patient, the method comprising:
using an assembly for closing the left atrial appendage; and
using a system for confirming closure of the left atrial appendage after the left atrial appendage has been closed by the assembly.

2. The method of claim 1, wherein:
the step of using the system includes:
introducing a fluorescence endoscopic dye at a position located proximate to left atrial appendage.

3. The method of claim 2, wherein:
the step of using the system includes:
activating a fluorescence excitation light after the fluorescence endoscopic dye has been introduced.

4. The method of claim 3, wherein:
the step of using the system includes:
detecting a presence of fluorescence associated after the fluorescence endoscopic dye is positioned in an area with normal blood flow in the heart.

5. The method of claim 3, wherein:
the step of using the system includes:
detecting a lack of fluorescence associated with a lack of fluorescence endoscopic dye positioned in the left atrial appendage, which provides confirmation of closure of the left atrial appendage.

6. A method for closing, and confirming closure of, the left atrial appendage of the heart of a patient, the method comprising:
using a lariat assembly for closing the left atrial appendage; and
using an epicardial fluorescence angiogram system for confirming closure of the left atrial appendage after the left atrial appendage has been closed.

7. The method of claim 6, wherein:
the step of using the epicardial fluorescence angiogram system includes introducing a fluorescence endoscopic dye, via an epicardial approach, at a position located proximate to left atrial appendage.

8. The method of claim 7, wherein:
the step of using the epicardial fluorescence angiogram system includes activating a fluorescence excitation light after the fluorescence endoscopic dye has been introduced to the left atrial appendage.

9. The method of claim 8, wherein:
the step of using the epicardial fluorescence angiogram system includes detecting a presence of fluorescence associated with the fluorescence endoscopic dye positioned in an area with normal blood flow in the heart.

10. The method of claim 8, wherein:
the step of using the epicardial fluorescence angiogram system includes detecting a lack of fluorescence associated with a lack of fluorescence endoscopic dye positioned in the left atrial appendage, which provides confirmation of closure of the left atrial appendage.

11. The method of claim 6, further comprising:
positioning a first medical guidewire assembly proximate to a first side of the left atrial appendage before using the lariat assembly for closing the left atrial appendage.

12. The method of claim 11, further comprising:
positioning a second medical guidewire assembly at a location proximate to a second side of the left atrial appendage, in which the second medical guidewire assembly is configured to be attracted to a first medical guidewire assembly positioned at the first side of the left atrial appendage in such a way that a portion of the left atrial appendage becomes securely held between distal end sections of the first medical guidewire assembly and the second medical guidewire assembly.

13. The method of claim 12, further comprising:
moving the lariat assembly toward the left atrial appendage for positioning the lariat assembly for secured connection to a section of the left atrial appendage after the left atrial appendage is securely held between the first medical guidewire assembly and the second medical guidewire assembly.

14. The method of claim 6, further comprising:
activating an expandable device in such a way that an interior of the left atrial appendage is supported by the expandable device, and the expandable device is deployed to ensure that the lariat assembly may be positioned for optimal installment of the lariat to the desired portion of the left atrial appendage before the lariat assembly is positioned for secured connection to a section of the left atrial appendage.

15. The method of claim 14, further comprising:
positioning the lariat assembly for secured connection to the section of the left atrial appendage after the expandable device is activated, the interior of the left atrial appendage is supported by the expandable device.

16. The method of claim 15, further comprising:
activating the lariat assembly for installment of a lariat to a desired portion of the left atrial appendage.

17. The method of claim 6, wherein:
activating the lariat assembly to apply an atrophication element that causes the left atrial appendage to become pinched, thereby initiating atrophication of the left atrial appendage.

18. The method of claim 6, wherein:
positioning a sensor device proximate to the left atrial appendage for viewing movement of the lariat assembly.
